(19) United States
(12) Patent Application Publication
(10) Pub. No.: US 2023/0181740 A1
(43) Pub. Date: Jun. 15, 2023

(54) VISCOSIFIED FLUIDS FOR MEDICAMENT DELIVERY

(71) Applicant: Palo Alto Research Center Incorporated, Palo Alto, CA (US)

(72) Inventors: Ravi NEELAKANTAN, San Francisco, CA (US); Jerome UNIDAD, San Francisco, CA (US)

(73) Assignee: Palo Alto Research Center Incorporated, Palo Alto, CA (US)

(21) Appl. No.: 17/551,289

(22) Filed: Dec. 15, 2021

(51) Int. Cl. A61K 47/38; A61K 9/10

(52) U.S. Cl. CPC A61K 47/38; A61K 9/10; B82Y 5/00

(57) ABSTRACT

Mass vaccination of one or more animals may be performed topically using viscosified fluids. Suitable viscosified fluids may comprise a viscosifying construct and a medicament admixed with an aqueous carrier fluid. The viscosifying construct comprises a polymer associated with a plurality of particles through an ionic interaction, covalent bonding, hydrogen bonding, or any combination thereof. The viscosified fluid may exhibit shear-thinning behavior and become sprayable once sheared. When applied to one or more animals and shear is no longer being applied, the viscosity may increase and the viscosifying construct may adhere the medicament upon a topical surface of the one or more animals.

### FIELD

The present disclosure generally relates to viscosified fluids as carriers for medicament delivery and methods for making and using the same.

### STATEMENT REGARDING FEDERALLY SPONSORED RESEARCH OR DEVELOPMENT

Not applicable.

### BACKGROUND

Topical delivery systems for medicaments require a careful selection of both the active component (medicament) and the delivery vehicle in which the active component is disposed. There is often a correlation between viscosity of the delivery vehicle and 1) the length of time over which the active component remains effective once applied topically, and 2) the ease with which the delivery vehicle may be applied topically. Low-viscosity topical delivery systems typically exhibit lower bioadhesion and may be easily lost from a topical surface. Topical delivery systems having higher viscosity may be held more robustly upon a topical surface, but they may be more difficult to apply due to their higher viscosity, either by spraying or direct manual application. Furthermore, some topical delivery systems may contain only a low level of active component, oftentimes due to poor compatibility between the active component and the delivery vehicle.

Uncontrolled disease spread within animal populations may have significant ecological, economic, and public health consequences. Disease spread within and across various animal species may promote evolutionary emergence of new disease strains. The new disease strains may be more transmissible and/or more virulent and pose a significant health hazard when spread from a “reservoir” animal species to humans or another animal species. Such cross-species zoonotic disease spread is believed to be responsible for a number of emerging infectious diseases such as Ebola, Lassa fever, swine flu, and COVID-19. Medicament delivery to animal populations, both in the wild and domestically, may help alleviate these factors and aid in stabilizing an animal population as well. For example, by slowing or stopping disease spread within an animal population, the overall health of the population may improve and increase survival of the population's members. Similarly, immunocontraception vaccination of animal populations may aid in managing overabundant populations of certain wildlife species.

Despite the benefits of administering medicaments to animal populations, there are issues of practicality associated with mass administration of medicaments, especially to animal populations in the wild. To facilitate mass administration of medicaments to an animal population, topical administration is often performed, such as by manual application or through spraying a suitable medicament delivery system. Spray delivery of medicaments may allow multiple animals within an animal population to be treated simultaneously. Once topically applied to an animal's skin, fur, feathers, scales, or the like, the medicament may be directly adsorbed systemically or it may be ingested and subsequently adsorbed as the animals groom themselves or each other. A difficulty with spray delivery approaches is that there are few delivery vehicles that provide a sufficiently low viscosity to promote sprayability, while simultaneously maintaining a high enough viscosity for topical retention (bioadhesion) to occur until the medicament is adsorbed or ingested. In addition, the varied temperature conditions at which animals live in the wild may lead to viscosity changes that result in premature loss of a medicament from an animal's skin, fur, feathers, scales, or the like before the medicament can exert its therapeutic effect, given the typical variance of viscosity with temperature. Thus, even if a medicament may be successfully sprayed upon an animal, the animal may move to a different location in its habitat, where the delivery vehicle decreases in viscosity and the medicament is lost from the animal. In addition, suitable delivery vehicles for mass administration of medicaments to animals also need to have sufficiently low toxicity for ingestion during grooming as well. Surfactants introduced to promote compatibility between a medicament and a delivery vehicle may also be problematic in some instances, as the surfactants may interfere with or inactivate lipid- or micelle-encapsulated medicaments.

In the case of adhering a medicament to an animal's skin, fur, feathers, scales or the like, a sticky, viscous delivery vehicle may be desirable. Example delivery vehicles of this type include glycerin jelly, which may facilitate medicament adhesion at mild temperatures ranging from 20° C. to about 25° C. However, at higher temperatures, glycerin jelly and similar viscous substances may significantly decrease in viscosity and become too runny for effective topical retention of medicaments to be realized. Conversely, at lower temperatures, such delivery vehicles may be too viscous to facilitate effective delivery through spraying. Indeed, even at idealized retention temperatures, the high viscosity of glycerin jelly and similar viscous substances may render spray delivery and retention of medicaments problematic. At the very least, the effective operating temperature range for such delivery vehicles may be rather narrow.

A particular wildlife disease of interest for mass administration of a medicament is white-nose syndrome (WNS), an emergent infectious disease in bat populations that is caused by the fungus Pseudogymnoascus destructans. WNS affects hibernating bats and is considered one of the worst wildlife diseases of modern times, as it has dramatically decreased bat populations. Particularly in the northeastern United States, where cases were first reported, bat population have been reduced up to 90% in some locations. Moreover, WNS continues to spread across the American continent, with cases now being reported on the West Coast. To date, effective treatments against the fungus that causes WNS have yet to be developed. Moreover, there remain issues with effectively administering vaccines or other medicaments en masse to a bat colony to combat WNS or other infectious diseases.

Given the importance of bats in many ecosystems, the decrease in bat populations is especially concerning. Moreover, the role of bats in spreading emerging zoonotic diseases (e.g., Ebola, Lassa fever, Marburg virus, and coronaviruses, such as COVID-19) in various ecological hot spots is also worrisome from a public health standpoint. Therefore, developing suitable delivery vehicles and techniques to facilitate mass vaccination or treatment of bats against disease may be desirable with respect to both ecological and public health standpoints. While bats are a specific example of a “reservoir” species implicated in zoonotic disease spread, there are other mammalian and non-mammalian species that are also of interest in regard to mass vaccination or treatment to preclude unwanted disease transfer to humans and other animal species.

### SUMMARY

In some aspects, the present disclosure provides viscosified fluids for medicament delivery. The viscosified fluids comprise: a viscosifying construct and a medicament admixed with an aqueous carrier fluid, the viscosifying construct comprising a polymer associated with a plurality of particles through an ionic interaction, covalent bonding, hydrogen bonding, or any combination thereof; wherein the viscosified fluid exhibits shear-thinning behavior and is sprayable once sheared.

In other aspects, the present disclosure provides methods for delivering a medicament to an animal. The methods comprise: providing a viscosified fluid comprising a viscosifying construct and a medicament admixed with an aqueous carrier fluid, the viscosifying construct comprising a polymer associated with a plurality of particles through an ionic interaction, covalent bonding, hydrogen bonding, or any combination thereof; forming a shear-thinned fluid by shearing the viscosified fluid to decrease a viscosity thereof; and applying the shear-thinned fluid topically upon at least one animal.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1 is a diagram of a network structure formed from a polymer and a plurality of particles, in which there is a direct interaction between the polymer and the particles.

FIG. 2 is a diagram of a network structure formed from a polymer and a plurality of particles, in which there is an indirect interaction between the polymer and the particles via an intermediate species.

### DETAILED DESCRIPTION

The present disclosure relates to viscosified fluids as carriers for medicament delivery and methods for making and using the same.

As discussed above, there are ongoing challenges associated with mass administration of medicaments to animal populations through topical delivery. Topical retention of a medicament upon an animal and low toxicity of a medicament delivery vehicle may be difficult to realize in combination with one another. In the case of mass medicament delivery by spraying, viscous substances that may promote adherence of a medicament to an animal's skin, fur, feathers, scales, or the like may be difficult to spray. Moreover, as animals move within their habitat, medicament loss may occur from a topical location due to a decrease in viscosity of the delivery vehicle upon an animal.

The present disclosure describes viscosified fluids that may provide biocompatibility, stability, bioadhesion, and convenient viscosity values for topical delivery of medicaments present therein. Advantageously, the viscosified fluids may exhibit shear-thinning behavior to facilitate delivery through spraying at a low viscosity while becoming viscous once applied to a topical location upon an animal (e.g. upon the animal's skin, fur, feathers, scales, or the like), thereby promoting retention of the medicament thereon. Fluids are shear-thinning if the viscosity decreases as the shear rate or mechanical force applied to the fluid increases. The decreased viscosity of a shear-thinning fluid may also be maintained for a period of time after shearing ceases. Moreover, the viscosified fluids of the present disclosure may remain viscous over a range of temperatures commonly encountered in animal habitats (e.g., up to about 40° C.), thus limiting medicament loss as an animal moves between different temperature conditions.

The viscosified fluids described herein may comprise a viscosifying construct that affords the foregoing features and may further provide additional advantages for medicament delivery to a topical location upon an animal. The viscosifying constructs disclosed herein may comprise a polymer and a plurality of particles associated in one or more ways with the polymer to provide sufficient viscosity for promoting topical delivery of a medicament. The viscosifying constructs may allow shear-thinning behavior to be realized, while promoting relatively high viscosity values when not being sheared (or following a recovery period after being sheared) and when exposed to the range of temperatures commonly encountered in animal habitats. Thus, the viscosifying constructs may facilitate medicament administration through spraying when the viscosity is low and ready retention at a topical location once the viscosity increases again following a defined time period. Advantageously, the viscosifying constructs disclosed herein may maintain their viscosity over a broad temperature range (when not being sheared or following a recovery period after shearing), thereby facilitating use in various types of animal habitats.

Furthermore, the viscosifying constructs disclosed herein may facilitate use of both the polymer and particles at concentrations lower than if either of these components were used individually to promote a similar degree of viscosification. That is, the polymer and the particles may interact synergistically to promote viscosification in the disclosure herein through formation of the viscosifying construct. By lowering the concentration of polymer and particles, the biological compatibility of the viscosified fluids may be increased, especially in comparison to other types of conventional viscosifiers. Alternately, the polymer and/or particles may be utilized in amounts below which toxicity of either component may otherwise become problematic. Advantageously, bio-based polymers having low toxicity, such as chitosan, may be utilized for forming viscosifying constructs in the disclosure herein. Chitosan may also be advantageous for its anti-fungal properties in some situations.

As indicated above, the viscosified fluids disclosed herein may be administered to an animal population by spraying. Spraying a medicament, such as a therapeutic agent (drug) or vaccine, may be desirable for mass treatment of an animal population, especially when the animal population is not easily accessible and/or the number of animals is too large to allow individual medicament administration to take place. Animal populations that may be treated with the viscosified fluids disclosed herein are not believed to be particularly limited and may include both domestic animals, such as livestock, and wild animal groups of various types. The wild animals may be mammals, such as bats, prairie dogs, rodents, squirrels, raccoons, cats, dogs, apes, monkeys, and other small or large mammals, birds, reptiles, or the like. Depending on the type of animal, the viscosifying construct and medicament may stick to the animal's skin, fur, feathers, scales, or the like when applied topically, such as through spraying. The medicament may then be ingested as the animals groom themselves or each other, after which it may impart a therapeutic effect. Alternately, systemic absorption of the medicament through the animal's skin may take place.

Advantageously, the viscosified fluids of the present disclosure may be surfactant-free in some instances. The viscosifying constructs alone may be sufficient to disperse a medicament in an aqueous carrier fluid. Avoidance of surfactants may be desirable when a medicament is liposome- or lipid-encapsulated, which may be disrupted in the presence of a surfactant. Bio-based medicaments may also be disrupted in the presence of a surfactant in some instances.

All numerical values within the detailed description and the claims herein are modified by “about” or “approximately” with respect to the indicated value, and take into account experimental error and variations that would be expected by a person having ordinary skill in the art. Unless otherwise indicated, ambient temperature (room temperature) is about 25° C.

As used in the present disclosure and claims, the singular article forms “a,” “an,” and “the” include plural forms unless the context clearly dictates otherwise.

The term “and/or” as used in a phrase such as “A and/or B” herein is intended to include “A and B,” “A or B,” “A,” and “B.”

For the purposes of the present disclosure, the new numbering scheme for groups of the Periodic Table is used. In said numbering scheme, the groups (columns) are numbered sequentially from left to right from 1 through 18.

Unless otherwise indicated, room temperature is 23° C.

As used herein, the term “shear” refers to stirring or a similar process that induces mechanical agitation to a fluid.

As used herein, the term “aqueous carrier fluid” refers to any fluid containing at least 50 wt. % water.

Viscosified fluids of the present disclosure may comprise a viscosifying construct and a medicament admixed with an aqueous carrier fluid. The viscosifying construct may comprise a polymer associated with a plurality of particles through an ionic interaction, covalent bonding, hydrogen bonding, or any combination thereof. The viscosified fluids may exhibit shear-thinning behavior, as discussed in further detail below. The viscosified fluids may become sprayable once sheared. Shearing may take place before spraying, or the shearing process may take place in concert with the spraying process in some instances.

Suitable medicaments may include any therapeutic agent that may be adsorbed through an animal's skin and/or ingested orally to exert a therapeutic effect. Medicaments may include drugs, such as an anti-inflammatory agent, an antibiotic, an antifungal agent, an antiviral agent, a chemotherapeutic agent, or the like, or a vaccine, for example. The medicament may exert a local or systemic effect when applied topically and subsequently adsorbed or once ingested.

The medicament may be an anti-inflammatory agent suitable to treat any inflammation condition, which may be either short-lived (acute) or long-lasting (chronic). Inflammatory responses maybe associated with multiple wildlife diseases having a range of origins including, for example, bacterial (e.g., Salmonella), viral (e.g., rabies) or fungal (e.g., white-nose syndrome). Suitable anti-inflammatory agents for animals, including small mammals (e.g., bats) will be familiar to one having ordinary skill in the art.

The medicament may be an antibiotic for treatment of a bacterial infection. Suitable antibiotics for animals, including small mammals (e.g., bats) will be familiar to one having ordinary skill in the art.

The medicament may be an antiviral agent. Nonlimiting examples of antiviral agents may include those designed to treat rabies, herpes viruses, coronaviruses (e.g., SARS-CoV-2, COVID-19, MERS and the like), hepatitis B and C, influenza viruses (e.g., influenza A, B, H1N1, and the like), and the like. Suitable antiviral agents for animals, including small mammals (e.g., bats) will be familiar to one having ordinary skill in the art.

The medicament may be an anticancer agent effective in the treatment of malignant or benign tumors. Suitable anticancer agents for animals, including small mammals (e.g., bats) will be familiar to one having ordinary skill in the art.

Medicaments that may be administered to small mammals, such as bats, to treat an existing medical condition (disease) may include, but are not limited to, antibiotics, anesthetics, analgesics, antifungals, antihelminthics, and broad spectrum antimicrobials. Representative examples of medicaments among these classes may include, for instance, amoxicillin, amoxicillin-clavulanic acid, enroflaxcin, cephalosporin, clindamycin, doxycycline, isoflurane, tiletamine/zolazepam (TELAZOL), diazepam, ketamine, benzocaine/butamben/tetracaine (CETACAINE), meloxicam, tramadol, buprenorphrine, grifulvin V (micro-sized griseofulvin), chlorhexidine acetate, chlorhexidine gluconate, praziquantel, sulfadimethoxine, fenbendazole, Manuka honey, hypochlorous acid, and the like.

The medicament may be a vaccine in some instances. Unlike other types of medicaments, vaccines may be administered to an animal to prevent a medical condition (disease) from occurring, rather than treating an existing disease. Suitable vaccines may be utilized to provide protection against any of the types of conditions specified above. That is, suitable vaccines may be selected to protect against an inflammatory condition, a bacterial condition, a fungal condition, a viral condition, a cancer, or any combination thereof. For example, the vaccine may be effective to treat white-nose syndrome (WNS) in bats in some instances. In various vaccine applications, the vaccine may induce immunity within a vaccinated animal by presenting key proteins or virions associated with a viral disease. In other instances, vaccines may promote immunity against non-viral diseases (e.g., bacterial infections such as Salmonella or fungal diseases such as WNS). This strategy may be critical for combating WNS in bats, as no antifungal agents have yet been identified that effectively combat the fungus that produces WNS once an infection has occurred.

Suitable vaccines that may be delivered using the viscosified fluids of the present disclosure include attenuated vaccines (vaccines containing live, attenuated microorganisms), inactivated vaccines (vaccines containing inactivated, but previously virulent, micro-organisms that have been destroyed with chemicals, heat, or radiation, with intact but empty bacterial cell envelopes), toxoid vaccines, subunit vaccines, conjugate vaccines, outer membrane vesicle (OMV) vaccines, heterologous vaccines, viral vector vaccines, RNA vaccines and other nucleic acid vaccines, recombinant vector vaccines, dendritic vaccines, protein unit or subunit vaccines, and the like. In more specific examples, the vaccine may be selected virus-vectored vaccines (e.g., lipid membrane virus vector, or non-lipid membrane virus vector), virus-like particles, free antigens, sub-unit antigens, liposome-formulated nucleic acid vaccine, and any combination thereof. For example, the medicament may comprise a vaccine (e.g., an adenovirus vector) encapsulated in a lipid layer.

The viscosified fluid may have a loading of the medicament of about 10 wt. % or less, or about 5 wt. % or less, or about 2 wt. % or less, or about 1 wt. % or less, based on total mass of the viscosified fluid. In the case of a vaccine as a medicament, the viscosified fluid may have a loading of the vaccine ranging from about 10⁴ PFU (plaque forming units) to about 10¹⁰ PFU per 100 μL of viscosified fluid. In non-limiting embodiments, the medicament may be present at a concentration such that it may be uniformly distributed, either as a dispersion or a solution in an aqueous medium (aqueous carrier fluid) containing the viscosifying construct. A minimum concentration of the medicament in the viscosified fluid may be a therapeutically effective amount sufficient to produce a physiological effect once applied to an animal.

The viscosified fluids may comprise an effective amount of the viscosifying construct to afford a viscosity that is sufficiently low (when sheared or during a post-shearing recovery period) to facilitate administration by spraying and that is sufficiently high (when not sheared or after a post-shearing recovery period) to promote bioadhesion to a topical location upon an animal (e.g., upon the animal's skin, fur, feathers, scales, or the like). In non-limiting embodiments, the viscosifying construct may be present in an aqueous carrier fluid at a concentration such that the plurality of particles is present below a percolation threshold of the particles alone. For both particles and polymer separately, the percolation threshold is defined by a critical concentration (c*), also known in the art as the overlap concentration, wherein adjacent particles contact each other. The critical concentration (c*) may depend upon the dimensions of the particle or the length of polymer chains. In the case of cellulose particles having a largest dimension of about 1 micron, the critical concentration may occur at a concentration of about 5 wt. % or less. By having the particles in the viscosifying construct present below the percolation threshold, potential toxic effects associated with higher particle concentrations may be lessened.

In non-limiting examples, the loading of polymer in the viscosified fluids may range from about 0.001 wt. % to about 30 wt. %, or about 0.001 wt. % to about 20 wt. %, or about 0.01 wt. % to about 20 wt. %, or about 0.1 wt. % to about 20 wt. %, or about 1 wt. % to about 20 wt. %, or about 1 wt. % to about 15 wt. %, or about 1.5 wt. % to about 10 wt. %, or about 2 wt. % to about 5 wt. %, based on the total mass of the viscosified fluids.

In non-limiting examples, the loading of particles in the viscosified fluids may range from about 0.005 wt. % to about 80 wt. %, or about 0.005 wt. % to about 75 wt. %, or about 0.005 wt. % to about 70 wt. %, or about 0.005 wt. % to about 65 wt. %, or about 0.005 wt. % to about 60 wt. %, or about 0.05 wt. % to about 60 wt. %, or about 0.5 wt. % to about 60 wt. %, or about 1 wt. % to about 60 wt. %, or about 2 wt. % to about 55 wt. %, or about 5 wt. % to about 50 wt. %, or about 10 wt. % to about 60 wt. %, or about 15 wt. % to about 55 wt. %, or about 20 wt. % to about 50 wt. %, or about 25 wt. % to about 45 wt. %, or about 30 wt. % to about 35 wt. %, based on the total mass of the viscosified fluids. Some of these particle loadings may be below the particle percolation threshold, as referenced above.

The viscosifying constructs of the present disclosure may have a mass ratio of particles to polymer ranging from about 1:10 to about 10:1, or about 1:10 to about 1:1, or about 1:5 to about 5:1, or about 1:1 to about 10:1.

The viscosifying constructs may be present in the viscosified fluids in an amount sufficient to produce a viscosity of at least about 10 Pa·s, such as from about 10 Pa·s to about 100 Pa·s, or about 15 Pas to about 90 Pa·s, or about 20 Pas to about 80 Pa·s, or about 25 Pa·s to about 70 Pa·s, or about 30 Pa·s to about 60 Pa·s at zero shear and at a temperature up to about 40° C. Higher viscosity values may be measured at lower temperatures. As used herein, viscosity of the viscosified fluids are measured at either 20° C. or 40° C. using strain-controlled shear rheometry, in which the rheometer is equipped with parallel plates.

The viscosified fluids of the present disclosure may exhibit shear-thinning behavior. Over a range of shear rates (e.g., 0.1 to 1000 s⁻¹), the viscosity of a shear-thinning fluid may decrease as a function of the shear rate. Depending on the shear rate and concentration of the viscosifying construct in the viscosified fluids, the viscosity after shearing may approach that of the aqueous carrier fluid alone. The recovery period during which the viscosity returns to its pre-shearing value may range from about 5 seconds to about 30 minutes, or about 10 seconds to about 15 minutes, or about 30 seconds to about 20 minutes, or about 1 minute to about 60 minutes.

The viscosifying constructs in the viscosified fluids disclosed herein may be formed at a pH of about 4 to about 6, such as from about 4.2 to about 5.8, or about 4.4 to about 5.6, or about 4.6 to about 5.4, or about 4.8 to about 5.2.

The viscosifying constructs formed from the polymer and the plurality of particles may comprise any extended network structure formed between the polymer and the particles, such as through an ionic interaction (e.g., a salt bridge interaction between a protonated amine and a carboxylate), covalent bonding, hydrogen bonding, or any combination thereof. Such interactions may occur directly between the polymer and the particles (i.e., between complementary functional groups upon the polymer and the particles) or indirectly through an intermediate species, which may associate with suitable functional groups upon the polymer and the particles, such as a crosslinking agent that forms covalent bonds or associates ionically with the polymer and/or the particles. An intermediate species may allow functional groups present upon a polymer and particles to become associated with one another in a network structure when the functional groups are otherwise not directly complementary with one another (e.g., when the same type of functional groups or functional groups having the same charge are present upon both the polymer and the particles).

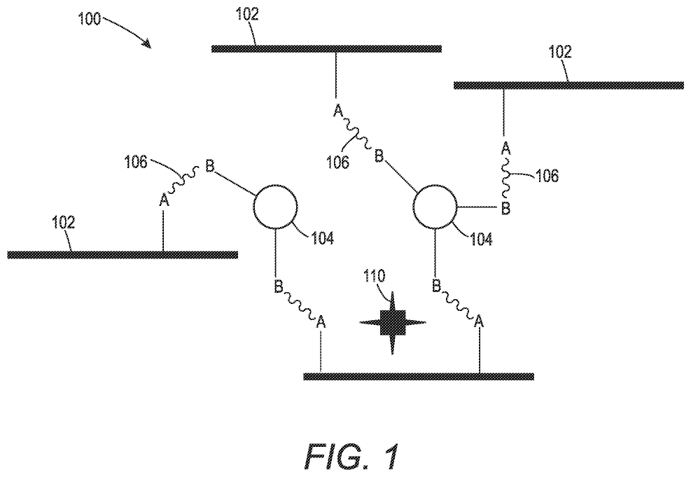

FIG. 1 is a diagram of a network structure formed from a polymer and a plurality of particles, in which there is a direct interaction between the polymer and the particles. As shown, viscosifying construct 100 includes polymers 102 containing functional group A and particles 104 containing functional groups B. Functional groups A and B are complementary to one another and may directly associate 106 with one another through an ionic interaction, covalent bonding, hydrogen bonding, or any combination thereof. Medicament 110 may be entrained within viscosifying construct 100 to promote delivery thereof.

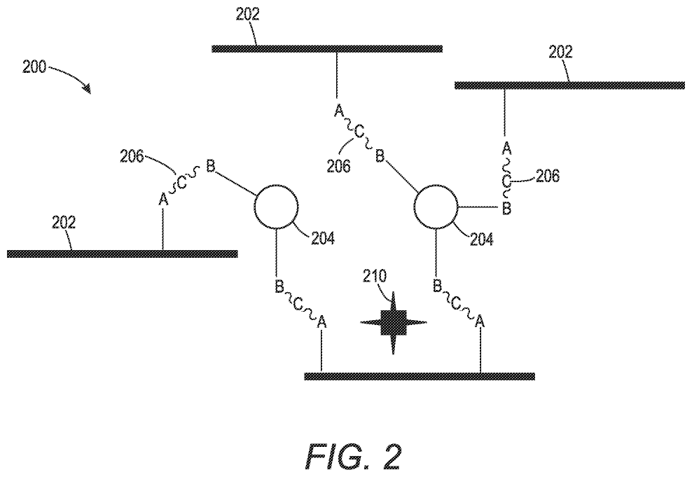

FIG. 2 is a diagram of a network structure formed from a polymer and a plurality of particles, in which there is an indirect interaction between the polymer and the particles via an intermediate species. As shown, viscosifying construct 200 includes polymers 202 containing functional group A and particles 204 containing functional groups B. Functional groups A and B may or may not be complementary to one another, but they are complementary to at least two functional groups (not shown) that are located upon intermediate species C. Intermediate species C may allow polymers 202 and particles 204 to associate 206 indirectly with one another through an ionic interaction, covalent bonding, hydrogen bonding, or any combination thereof. For example, intermediate species C may be bifunctional and comprise functional group A′ that is complementary with functional group A and functional group B′ that is complementary with functional group B. Medicament 210 may be entrained within viscosifying construct 200 to promote delivery thereof.

In some embodiments, the polymer and the particles may comprise nucleophilic or electrophilic functional groups, wherein a first functional group upon the polymer is nucleophilic or electrophilic and a second functional group upon the particles is the other of nucleophilic or electrophilic. For example, if the polymer has nucleophilic functional groups such as amines, the particles may comprise electrophilic functional groups, such as N-hydroxysuccinimides, to promote covalent bond formation. Likewise, if the polymer has electrophilic functional groups such as sulfosuccinimides, the particles may have nucleophilic functional groups such as amines, thiols, or carboxylic acids.

In some embodiments, the polymer and the particles may comprise hydrogen bond donors or hydrogen bond acceptors. For example, a first functional group upon the polymer may be a hydrogen bond donor, and a second functional group upon the particles may be a hydrogen bond acceptor.

The polymer and the particles may comprise functional groups with opposite charges, which may promote salt bridge formation or a similar type of ionic interaction. More specifically, the polymer and the particles may be associated with each other through an ionic interaction in which the first functional group and the second functional group bear opposite charges. In more particular examples, the polymer may be positively charged and the particles may be negatively charged. For example, the polymer may comprise positively charged amine groups, and the particles may comprise negatively charged sulfate groups, sulfonate groups, and/or carboxylate groups. Thus, functional polymers such as proteins, poly(allyl amine), or amine-terminated di- or multifunctional poly(ethylene glycol) (PEG) may be used as the polymer in the disclosure herein in combination with particles bearing sulfate, sulfonate, carboxylate, or phosphate groups. Other suitable examples of polymers and particles are described further below.

Particles suitable for incorporation in the viscosifying constructs are not believed to be particularly limited, provided that they are biocompatible and contain suitable functional groups for pairing with those upon a polymer or an intermediate species. Suitable particles may include, for example, include minerals, silicates, synthetic clays, biopolymers, and any combination thereof. The particles may be native or have undergone chemical modification (e.g., thermal reduction, hydrolysis, sulfonation, or the like). The particles may be isotropic (e.g., true particles, spheres, or precipitates) or anisotropic (e.g., platelets, rods, fibrils, or the like). The particles may be of any size range and may comprise nanoparticles in particular embodiments. Suitable sizes may range from about 1 nm up to about 1 micron in a largest dimension of the particles.

In particular embodiments, the particles may comprise cellulose nanoparticles (CNP) that are functionalized with at least one acid group. CNP (e.g., cellulose nanocrystals) are a class of bio-based nanoscale materials, which are of interest due to their biocompatibility, biodegradability, and renewability. CNP may be categorized into two major classes, (a) nanostructured materials (including cellulose microcrystals and cellulose microfibrils) and (b) nanofibers (including cellulose nanofibrils, cellulose nanorods, cellulose nanocrystals, and bacterial cellulose).

Cellulose nanoparticles, such as cellulose nanocrystals, suitable for use in the disclosure herein may be prepared by any suitable technique known to persons having ordinary skill in the art. Cellulose nanoparticles bearing at least one acid group may be prepared by acid hydrolysis of cellulose, followed by freeze-drying. The cellulose may be obtained from any suitable source, such as cotton or biomass. Hydrolysis using sulfuric acid may introduce sulfate groups to the surface of the cellulose nanoparticles, thereby facilitating their use in the disclosure herein, as well as permitting their dispersion in both aqueous and organic fluids, including water, ethanol and N,N-dimethylformamide. Carboxylate groups may be introduced through oxidation to the surface of the cellulose nanoparticles in some instances.

Alternately, the surface of cellulose nanoparticles may be modified with various electrophiles to promote covalent bond formation with a nucleophilic functional group upon the polymer. For example, the cellulose nanoparticles may be modified to introduce electrophilic α-bromoesters to the particle surface, which may then undergo a reaction with a nucleophile to promote direct covalent bond formation with a complementary functional group upon the polymer (e.g., a thiol, an amine, a carboxyate, a phosphine, or the like) that may vary in hydrophobicity, functionality, and complexity.

Suitable particles for use in the disclosure herein may have a maximum size of about 1 micron in a largest dimension thereof. For example, fibrils may be about 1 micron in length but have other dimensions that are much smaller, such as about 100 nm or less in other dimensions. Particle size can be determined by scanning electron microscopy or dynamic light scattering techniques.

In some instances, the polymer may comprise an amine-containing carbohydrate, such as chitosan. Chitosan has a carbohydrate backbone structure similar to cellulose, which includes two types of repeating units, N-acetyl-D-glucosamine and D-glucosamine, linked by (1-4)-β-glycosidic linkages. Chitosan has been widely used in the pharmaceutical industry due to its biodegradability and biocompatibility properties. Chitosan is a cationic natural polysaccharide exhibiting antimicrobial and antifungal activities, which makes it a favorable excipient option for biomedical applications. Viscosified fluids containing a viscosifying construct formed from chitosan may be especially advantageous for treating white-nose syndrome due to the fungal nature of this condition. In addition, the amine groups of chitosan provide complementary functionality for association via an ionic interaction with negatively charged functional groups upon particles, such as cellulose nanoparticles functionalized with an acidic group, when forming the viscosifying constructs disclosed herein.

Chitosan may be obtained through deacetylation of chitin, often obtained from the shells of shrimp and other sea crustaceans, using excess aqueous sodium hydroxide. Chitosan is soluble in dilute acidic solutions of acetic, citric, and tartaric acids at a pH less than about 6.5, with solubility at about 1-3 wt. % being realized through protonation of the amine group. Suitable chitosans for forming a viscosifying construct may have molecular weight values (Mw) ranging from about 3,800 to about 190,000 Da and with different degrees of deacetylation.

Depending on the functional groups present on the particles, chitosan derivatives may also be suitable for use in the disclosure herein. Suitable chitosan derivatives may include N-alkyl chitosans, thiolated chitosans, carboxylated chitosans, chitosan succinate, chitosan phthalate, mono-N-carboxymethyl chitosan (MCC), or any combination thereof. Other carbohydrate derivatives bearing suitable functionality for interacting with a given functional group upon the particles may also be suitably used in the disclosure herein.

Other types of polymers may also be used in the disclosure herein, provided that they contain or are derivatized to contain one or more functional groups that are complementary with one or more functional groups upon the particles. Suitable alternative polymers may include medical grade synthetic polymers such as, but not limited to, amino-acid-based polymers, polyamides, polyimides, polyurethanes, cellulose-based polymers, starch-based polymers, water-insoluble biodegradable polymers, alginates and other hydrocolloid-based polymers, and any combination thereof. Biocompatible polyamides and polyurethanes may be polyamides and polyurethanes containing a phospholipid moiety.

Other examples of suitable polymers that may be used in the disclosure herein include, for example, poly(ethylene glycol) (PEG), poly(propylene glycol) (PEG), polylactic acid (PLA), polyglycolic acid (PGA), poly (DL-lactide-co-glycolide) (PLG), polyanhydrides, polyacrylic acid, polymethyl acrylates, poly(ε-caprolactone) (PCL), poly(3-hydroxybutyrate-co-3-hydroxyvalerate) (PHBV), any copolymer thereof, or any combination thereof.

A first functional group upon the polymer and a second functional group upon the particles may be associated with each other through an intermediate species that is a crosslinking agent. Polymers may be crosslinked with a plurality of particles via an ionic interaction, a covalent bond, a hydrogen bond, or any combination thereof. A crosslinking agent may be employed when a first functional group upon the polymer is not directly complementary with a second functional group upon the particles. The crosslinking agent may be bifunctional and bear at least two types of functional groups that are complementary with the first functional group upon the polymer and the second functional group upon the particles, thereby allowing a network structure to form effectively in the presence of the crosslinking agent. Nonlimiting examples of suitable crosslinking agents may include transition metal salts, borates, boronic acids, bifunctional crosslinkers, and any combination thereof, provided that the crosslinking agent is sufficiently biocompatible once incorporated in a viscosifying construct. A crosslinking agent may facilitate association between a polymer and particles that have the same charge or a different charge, or between a charged polymer and uncharged particles or vice versa.

When used, crosslinking agents may be present in the viscosified fluids disclosed herein in an amount ranging from about 5 wt. % to about 70 wt. %, or about 10 wt. % to 60 wt. %, or about 15 wt. % to 50 wt. %, or about 20 wt. % to 45 wt. %, based on total mass of the viscosifed fluid.

Borate crosslinking agents may facilitate crosslinking between alcohols and amines. More particularly, suitable borate crosslinking agents may form borate ester bonds with alcohol functional groups, and the amine group may form a B—N bond through donation of a lone pair of electrons to the unoccupied p-orbital upon the boron atom. Examples of borate crosslinking agents may include, but are not limited to, boric acid, alkali or alkaline earth metal borates, such as disodium octaborate tetrahydrate or sodium diborate, and organic borates such as boronic acids and esters.

Suitable bifunctional crosslinking agents may contain the same functional group (if the first and second functional groups upon the polymer and the particles are the same) or different functional groups (if the first and second functional groups upon the polymer and particles are different). Suitable bifunctional crosslinking agents that may enable linkage via a thioether bond include N-succinimidyl-4-(N-maleimidomethyl)-cyclohexane-1-carboxylate (SMCC) to introduce maleimido groups, or with N-succinimidyl-4-(iodoacetyl)-aminobenzoate (SIAB) to introduce iodoacetyl groups. Other bifunctional crosslinking agents that may introduce maleimido groups or haloacetyl groups include, but not limited to, bis-maleimidopolyethyleneglycol (BMPEO), BM(PEO)₂, BM(PEO)₃, N-(3-maleimidopropyloxy)succinimide ester (BMPS), γ-maleimidobutyric acid N-succinimidyl ester (GMBS), ε-maleimidocaproic acid N-hydroxysuccinimide ester (EMCS), 5-maleimidovaleric acid NHS, HBVS, N-succinimidyl-4-(N-maleimidomethyl)-cyclohexane-1-carboxy-(6-amidocaproate), which is a “long chain” analog of SMCC (LC-SMCC), m-maleimidobenzoyl-N-hydroxysuccinimide ester (MBS), 4-(4-N-maleimidophenyl)-butyric acid hydrazide or HCl salt (MPBH), N-succinimidyl 3-(bromoacetamido)propionate (SBAP), N-succinimidyl iodoacetate (SIA), κ-maleimidoundecanoic acid N-succinimidyl ester (KMUA), N-succinimidyl 4-(p-maleimidophenyl)-butyrate (SMPB), succinimidyl-6-(3-maleimidopropionamido)hexanoate (SMPH), succinimidyl-(4-vinylsulfonyl)benzoate (SVSB), dithiobis-maleimidoethane (DTME), 1,4-bis-maleimidobutane (BMB), 1,4 bismaleimidyl-2,3-dihydroxybutane (BMDB), bis-maleimidohexane (BMH), bis-maleimidoethane (BMOE), sulfosuccinimidyl 4-(N-maleimido-methyl)cyclohexane-1-carboxylate (sulfo-SMCC), sulfosuccinimidyl(4-iodo-acetyl)aminobenzoate (sulfo-SIAB), m-maleimidobenzoyl-N-hydroxysulfosuccinimide ester (sulfo-MBS), N-(γ-maleimidobutryloxy)sulfosuccinimde ester (sulfo-GMBS), N-(ε-maleimidocaproyloxy)sulfosuccimido ester (sulfo-EMCS), N-(κ-maleimidoundecanoyloxy)sulfosuccinimide ester (sulfo-KMUS), and sulfosuccinimidyl 4-(p-maleimidophenyl)butyrate (sulfo-SMPB).

Heterobifunctional crosslinking agents are bifunctional crosslinking agents having two different reactive groups. Heterobifunctional crosslinking agents may contain both an amine-reactive N-hydroxysuccinimide group (NHS group) and a carbonyl-reactive hydrazine group. Examples of commercially available heterobifunctional crosslinking agents may include, but are not limited to, succinimidyl 6-hydrazinonicotinamide acetone hydrazone (SANH), succinimidyl 4-hydrazidoterephthalate hydrochloride (SHTH) and succinimidyl hydrazinium nicotinate hydrochloride (SHNH). Examples of bifunctional crosslinking agents that can be used include succinimidyl-p-formyl benzoate (SFB) and succinimidyl-p-formylphenoxyacetate (SFPA).

Bifunctional crosslinking agents that enable a linkage via disulfide bonds may include N-succinimidyl-3-(2-pyridyldithio)propionate (SPDP), N-succinimidyl-4-(2-pyridyldithio)pentanoate (SPP), N-succinimidyl-4-(2-pyridyldithio)butanoate (SPDB), N-succinimidyl-4-(2-pyridyldithio)2-sulfo butanoate (sulfo-SPDB) to introduce dithiopyridyl groups. Alternatively, crosslinking agents such as 2-iminothiolane, homocysteine thiolactone or S-acetylsuccinic anhydride that introduce thiol groups can also be used.

Suitable crosslinking agents may further include diallyl fumarate, diallyl diglycol carbonate, allyl methacrylate, diallyl phthalate, diallyl suverate, diallyl tetrabromophthalate, diethylene glycol diacrylate, diethylene glycol dimethacrylate, diethylene glycol divinyl ether, N, N′-dimethacryloylpiperazine, 2,2-dimethylpropanediol dimethacrylate, dipentaerythritol pentaacrylate, dipropylene glycol dimethacrylate, ditrimethylolpropane tetraacrylate, divinyl glycol, divinyl sebacate, glycerol trimethacrylate, 1,5-hexadiene, 1,6-hexanediol diacrylate, 1,6-hexane Diol diacrylate, 1,6-hexanediol dimethacrylate, N,N′-methylenebismethacrylamide, 1,9-nonanediol dimethacrylate, pentaerythritol tetraarc Relate, pentaerythritol triacrylate, pentaerythritol triallyl ether, 1,5-pentanediol dimethacrylate, poly (propylene glycol) dimethacrylate, tetraethylene glycol dimethacrylate, triethylene glycol di Acrylate, triethylene glycol dimethacrylate, triethylene glycol divinyl ether, 1,1,1-tri methylol ethane trimethacrylate, 1,1,1-trimethylolpropane diallyl ether, 1,1,1-Trimethylolpropane triacrylate, 1,1,1-trimethylolpropane trimethacrylate, tripropylene glycol diacrylate, 1,2,4-trivinylcyclohexane, divinyl benzene, bis (2-methacryl oxyethyl) phosphate, 2,2-bis (4-methacryloxyphenyl) propane, 1,3-butanediol diacrylate, 1,4-butanediol diacrylate, 1,3-butanediol dimethacrylate, 1, 4-butanediol dimethacryl Rate, 1,4-butanediol divinyl ether, 1,4-cyclohexanediol dimethacrylate, bis [4-(vinyloxy) butyl] isophthalate, bis [4-(vinyloxymethyl) cyclohexylmethyl] glycol Rutarate, bis [-(vinyloxy) butyl] succinate, bis ((4-((vinyloxy) methyl) cyclohexyl) methyl) isophthalate, bis (4-(vinyloxy) butyl) terephthalate, bis [[(4-(vinyloxy) methyl) cyclohexyl] methyl] terephthalate, bis [4-(vinyloxy) butyl] adipate, bis [4-(vinyloxy) butyl] (methylenedi-1,4-phenylene) biscarbamate, bis [4-(vinyloxy) butyl] (4-methyl-1,3-phenylene) biscarbamate, bis [4-(vinyloxy) butyl] 1,6-hexanediylbis Carbamate or tris [4-(vinyloxy) butyl] trimelitate.

As discussed above, a first functional group upon the polymer may be directly associated with a second functional group upon the particles. In this case, the polymer or particles act as a crosslinker. Hence, when a crosslinking agent or other intermediate species is absent, the functional groups are oppositely charged or otherwise complementary to form a covalent bond or hydrogen bonding interaction.

The viscosified fluids of the present disclosure may further include stabilizing agents, surfactants and/or adjuvants. Examples surfactants may be any non-toxic, biocompatible and biodegradable surfactants. Examples of suitable surfactants may include, but are not limited to, zwitterionic, neutral, cationic, or anionic surfactants. Common hydrophilic head groups of ionic or zwitterionic surfactants are carboxylate, sulfate, sulfonate, carboxybetaine, sulfobetaine, and quaternary ammonium. Suitable neutral surfactants may include, but are not limited to, lipid-based surfactants, polyethylene glycol (PEG) block-copolymer surfactants, polysorbates, such as TWEEN 20 and TWEEN 80, other sorbitan derivatives, such as sorbitan laurate, and alkoxylated alcohols, such as laureth-4.

Alternately, the viscosified fluids of the present disclosure may be surfactant-free. A surfactant-free viscosified fluid may be desirable when the surfactant interacts in a medicament in an undesired way. For example, surfactants may change the conformation of protein-based medicaments or interfere with a lipid- or micelle-encapsulated medicament.

Methods of the present disclosure may comprise: providing a viscosified fluid comprising a viscosifying construct, and a medicament admixed with an aqueous carrier fluid, and applying the viscosified fluid to an animal in need of treatment for a medical condition. As described above, the viscosifying construct may comprise a polymer associated with a plurality of particles through an ionic interaction, covalent bonding, hydrogen bonding, or any combination thereof. Once provided, the viscosified fluid may be then sheared to decrease its viscosity thereof, thereby forming a shear-thinned fluid. The shear-thinned fluid may then be applied topically upon at least one animal, such as through spraying. Once applied topically to the animal and shear is no longer being applied, the fluid may regain its viscosity and stick to the skin, fur, feathers, or scales of the animal, depending on the type of animal being treated, thereby facilitating medicament delivery and retention. The medicament may be absorbed through the skin of the animal, or preferably ingested by the animal as the animal grooms itself or a group of animals grooms each other.

In some embodiments, the animal may be at least one mammal. Mammals that may be treated with the viscosified fluids described herein are not believe to be particularly limited and may include, for example, bats, prairie dogs, rodents, raccoons, livestock animals (e.g., cows, horses, goats, sheep, and the like) cats, dogs, apes, and other small or large mammals, wild or domesticated. Preferably, the mammal has fur, the viscosifying construct and the medicament may stick to the fur of the mammal once the shear-thinned fluid has been applied thereto.

Once formed, the shear-thinned fluid may be applied to individual animals or to multiple animals simultaneously. To facilitate deliver to multiple animals simultaneously, the shear-thinned fluid may be applied topically through spraying in various embodiments. Suitable spraying techniques are not believed to be particularly limited. Alternately, manual application techniques may be used.

In a particular example, methods for applying a surfactant-free viscosified fluid that has anti-fungal properties may comprise: providing a viscosified fluid comprising a viscosifying construct and a medicament admixed with an aqueous carrier fluid, wherein the viscosifying construct comprises chitosan associated with a plurality of cellulose nanoparticles functionalized with at least one acid group, and the medicament is a sprayable vaccine; forming a shear-thinned fluid by shearing the viscosified fluid to decrease a viscosity thereof; and spraying the shear-thinned fluid topically upon at least one animal, preferably at least one mammal. The viscosifying construct may be present in an amount sufficient to produce a viscosity of at least about 10 Pa·s at zero shear and at a temperature up to about 40° C.

Embodiments disclosed herein include:

A. Viscosified fluids for medicament delivery. The viscosified fluids comprise: a viscosifying construct and a medicament admixed with an aqueous carrier fluid, the viscosifying construct comprising a polymer associated with a plurality of particles through an ionic interaction, covalent bonding, hydrogen bonding, or any combination thereof; wherein the viscosified fluid exhibits shear-thinning behavior and is sprayable once sheared.

B. Methods for delivering a medicament. The methods comprise: providing a viscosified fluid comprising a viscosifying construct and a medicament admixed with an aqueous carrier fluid, the viscosifying construct comprising a polymer associated with a plurality of particles through an ionic interaction, covalent bonding, hydrogen bonding, or any combination thereof; forming a shear-thinned fluid by shearing the viscosified fluid to decrease a viscosity thereof; and applying the shear-thinned fluid topically upon at least one animal.

Embodiments A and B may have one or more of the following additional elements in any combination:

Element 1: wherein the polymer and the plurality of particles comprising the viscosifying construct are present in the aqueous carrier fluid at a concentration of about 5 vol. % or below.

Element 2: wherein the viscosifying construct is present in the aqueous carrier fluid in an amount sufficient to produce a viscosity of at least about 10 Pa·s at zero shear and at a temperature up to about 40° C., as determined by steady shear rheometry.

Element 3: wherein a first functional group upon the polymer and a second functional group upon the particles are associated with each other indirectly through an intermediate species.

Element 4: wherein a first functional group upon the polymer is directly associated with a second functional group upon the particles.

Element 5: wherein the polymer and the particles are directly associated with each other through an ionic interaction in which the first functional group and the second functional group have opposite charges.

Element 6: wherein the polymer and the particles are directly associated with each other through an ionic interaction in which the polymer is positively charged and the particles are negatively charged.

Element 7: wherein the polymer comprises an amine-containing carbohydrate.

Element 8: wherein the amine-containing carbohydrate comprises chitosan and the particles comprise cellulose nanoparticles, the cellulose nanoparticles being functionalized with at least one acid group.

Element 9: wherein the viscosifying construct forms at a pH of about 4 to about 6.

Element 10: wherein the particles have a maximum size of about 1 micron in a largest dimension thereof.

Element 11: wherein the viscosified fluid is surfactant-free.

Element 12: wherein the medicament comprises a vaccine.

Element 13: wherein the vaccine is selected from the group consisting of a virus-vectored vaccine, a virus-like particle, a free antigen, a sub-unit antigen, a liposome-formulated nucleic acid vaccine, and any combination thereof.

Element 14: wherein the shear-thinned fluid is applied upon multiple animals simultaneously.

Element 15: wherein the shear-thinned fluid is applied topically by spraying.

Element 16: wherein the at least one animal comprises at least one mammal.

By way of non-limiting example, illustrative combinations applicable to A and B include, but are not limited to, 1 and/or 2, and 3; 1 and/or 2, and 4; 1 and/or 2, and 5; 1 and/or 2, and 6; 1 and/or 2, and 7; 1 and/or 2, and 7 and 8; 1 and/or 2, and 9; 1 and/or 2, and 10; 1 and/or 2, and 11; 1 and/or 2, and 12; 1 and/or 2, and 12 and 13; 3 or 4 and 5; 3 or 4 and 6; 3 or 4 and 7; 3 or 4 and 7 and 8; 3 or 4 and 9; 3 or 4 and 10; 3 or 4 and 11; 3 or 4 and 12; 3 or 4 and 12 and 13; 5 or 6 and 7; 5 or 6 and 7 and 8; 5 or 6 and 9; 5 or 6 and 10; 5 or 6 and 11; 5 or 6 and 12; 5 or 6 and 12 and 13; 7 and 8; 7 and 9; 7 and 10; 7 and 11; 7 and 12; 7, 12 and 13; 8 and 9; 8 and 10; 8 and 11; 8 and 12; 8, 12 and 13; 9 and 10; 9 and 11; 9 and 12; 9, 12 and 13; 10 and 11; 10 and 12; 10, 12 and 13; 11 and 12; and 11-13. Any of the foregoing or any one of 1-13 may be in further combination with 14, 15, and/or 16. Additional exemplary combinations applicable to B include, but are not limited to, 14 and 15; 14 and 16; 14-16; and 15 and 16.

To facilitate a better understanding of the present disclosure, the following examples of preferred or representative embodiments are given. In no way should the following examples be read to limit, or to define, the scope of the invention.

### Examples

Viscosified fluids were prepared as specified in Table 1, and viscosity values were determined at 20° C. and 40° C. using shear rheometry.

TABLE 1

| | Viscosity | Viscosity |
|---|---|---|
| Formulation | (20° C.) | (40° C.) |
| 2 wt. % cellulose | 0.36 | 0.24 |
| nanocrystals in water | | |
| 2 wt. % chitosan in | 1.24 | 0.54 |
| water | | |
| 1 wt. % cellulose | 2.49 | 1.56 |
| nanocrystals/1 wt. % | | |
| chitosan in water | | |

As shown, the viscosity values were higher when particles and polymer were combined together in a viscosifying construct, in comparison to a higher concentration of particles or polymer alone. The viscosification effect was more than additive at equivalent total concentrations of polymer and particles.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

One or more illustrative embodiments are presented herein. Not all features of a physical implementation are described or shown in this application for the sake of clarity. It is understood that in the development of a physical embodiment of the present disclosure, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for one of ordinary skill in the art and having benefit of this disclosure.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

What is claimed is the following:

1. A viscosified fluid comprising:
a viscosifying construct and a medicament admixed with an aqueous carrier fluid, the viscosifying construct comprising a polymer associated with a plurality of particles through an ionic interaction, covalent bonding, hydrogen bonding, or any combination thereof;
wherein the viscosified fluid exhibits shear-thinning behavior and is sprayable once sheared.

2. The viscosified fluid of claim 1, wherein the polymer and the plurality of particles comprising the viscosifying construct are present in the aqueous carrier fluid at a concentration of about 5 vol. % or below.

3. The viscosified fluid of claim 1, wherein the viscosifying construct is present in the aqueous carrier fluid in an amount sufficient to produce a viscosity of at least about 10 Pas at zero shear and at a temperature up to about 40° C., as determined by steady shear rheometry.

4. The viscosified fluid of claim 1, wherein a first functional group upon the polymer and a second functional group upon the particles are associated with each other indirectly through an intermediate species.

5. The viscosified fluid of claim 1, wherein a first functional group upon the polymer is directly associated with a second functional group upon the particles.

6. The viscosified fluid of claim 5, wherein the polymer and the particles are directly associated with each other through an ionic interaction in which the first functional group and the second functional group have opposite charges.

7. The viscosified fluid of claim 5, wherein the polymer and the particles are directly associated with each other through an ionic interaction in which the polymer is positively charged and the particles are negatively charged.

8. The viscosified fluid of claim 7, wherein the polymer comprises an amine-containing carbohydrate.

9. The viscosified fluid of claim 8, wherein the amine-containing carbohydrate comprises chitosan and the particles comprise cellulose nanoparticles, the cellulose nanoparticles being functionalized with at least one acid group.

10. The viscosified fluid of claim 7, wherein the viscosifying construct forms at a pH of about 4 to about 6.

11. The viscosified fluid of claim 1, wherein the particles have a maximum size of about 1 micron in a largest dimension thereof.

12. The viscosified fluid of claim 1, wherein the viscosified fluid is surfactant-free.

13. The viscosified fluid of claim 1, wherein the medicament comprises a vaccine.

14. The viscosified fluid of claim 13, wherein the vaccine is selected from the group consisting of a virus-vectored vaccine, a virus-like particle, a free antigen, a sub-unit antigen, a liposome-formulated nucleic acid vaccine, and any combination thereof.

15. A method comprising:
providing a viscosified fluid comprising a viscosifying construct and a medicament admixed with an aqueous carrier fluid, the viscosifying construct comprising a polymer associated with a plurality of particles through an ionic interaction, covalent bonding, hydrogen bonding, or any combination thereof;
forming a shear-thinned fluid by shearing the viscosified fluid to decrease a viscosity thereof; and
applying the shear-thinned fluid topically upon at least one animal.

16. The method of claim 15, wherein the shear-thinned fluid is applied upon multiple animals simultaneously.

17. The method of claim 15, wherein the shear-thinned fluid is applied topically by spraying.

18. The method of claim 15, wherein the at least one animal comprises at least one mammal.

19. The method of claim 18, wherein the at least one mammal comprises a mammal selected from the group consisting of a bat, a prairie dog, a raccoon, a livestock animal, a cat, and a dog.

20. The method of claim 15, wherein the medicament comprises a vaccine.

21. The method of claim 20, wherein the vaccine is selected from the group consisting of a virus-vectored vaccine, a virus-like particle, a free antigen, a sub-unit antigen, a liposome-formulated nucleic acid vaccine, and any combination thereof.

22. The method of claim 16, wherein a first functional group upon the polymer is directly associated with a second functional group upon the particles.

23. The method of claim 22, wherein the polymer and the particles are directly associated with each other through an ionic interaction in which the first functional group and the second functional group have opposite charges.

24. The method of claim 22, wherein the polymer and the particles are directly associated with each other through an ionic interaction in which the polymer is positively charged and the particles are negatively charged.

25. The method of claim 24, wherein the polymer comprises an amine-containing carbohydrate.

26. The method of claim 25, wherein the amine-containing carbohydrate comprises chitosan and the particles comprise cellulose nanoparticles, the cellulose nanoparticles being functionalized with at least one acid group.
